(19) United States
(12) Patent Application Publication
(10) Pub. No.: US 2003/0161899 A1
(43) Pub. Date: Aug. 28, 2003

(54) Nasal ointment based on white petroleum jelly

(76) Inventor: Peter Theiss, Homburg/Saar (DE)

Correspondence Address: Michael R Ward, Morrison & Foerster, 425 Market Street, San Francisco, CA, 94105-2482, US

(21) Appl. No.: 10/296,934

(22) PCT Filed: May 31, 2001
(86) PCT No.: PCT/EP01/06197

(30) Foreign Application Priority Data
May 31, 2000 (DE) 200 09 841.1

(51) Int. Cl. A61K 35/78; A61K 31/695; A61K 31/198; A61K 31/01

(52) U.S. Cl. 424/735; 514/762; 424/764; 424/750; 424/765; 514/63; 514/561

(57) ABSTRACT

Nose ointment for the prophylaxis of inhalation allergic reactions comprising at least one mixture of saturated hydrocarbons characterized in that the at least one mixture of saturated hydrocarbons has a viscosity by the DIN 51 562 method of >6 mm²/s (100° C.).

The present invention relates to a nose ointment comprising mixtures, which are gel-like at room temperature, predominantly of saturated hydrocarbons, and to the use thereof for the prophylaxis of inhalation allergic reactions, in particular of atopic rhinitis in the form of “hayfever”. In a preferred embodiment, the nose ointment according to the invention also comprises at least one care additive suitable for ointments.

In the western industrialized civilization, inhalation allergies are now widespread, with a continuously increasing trend. These are, in particular, the forms of atopic rhinitis which are induced by inhalation of pollen, fungal spores, various dusts (for example of wood, flour or house dust), and chemical or animal (feathers, hair) irritants and which, with their side effects, may impair the patient's wellbeing as far as inability to work.

The paining itching of the nose, pharynx, back of the throat and eyes, which are followed by eye-watering, sneezing and watery discharge from the nose, is frequently accompanied by headache, irritability, loss of appetite, depression and insomnia. Subsequently, attacks of coughing and asthmatic wheezing may develop.

As a seasonal reaction induced by pollen from trees, shrubs, grasses as well as wild or ornamental plants, so-called “hayfever” is the principal form of atopic rhinitis.

Most of the therapeutic and prophylactic measures used up to now to control or eliminate inhalation allergic reactions show more or less serious disadvantages in relation to cost, efficiency, side effects on health and convenience of use.

Thus, cures entailing the patients being removed from the exposure area to areas low in pollen, such as high mountains or islands with little vegetation, for several weeks during the pollen season, or else a change in habitation or occupation to avoid exposure, can be afforded by many patients only with difficulty.

By this waiting period it is attempted to remove as far as possible the allergens from the patient's environment.

Complete removal of inhaled allergens is impossible. There is merely a chance of eliminating the contact surfaces to the allergen, that is to say the mucous membrane as site of reaction. This is described as partial avoidance of exposure.

The fitting of allergen filters in ventilation and air-conditioning systems in habitations, workplaces and cars is able to prevent exposure only in these specially protected areas and is likewise associated with non-negligible costs.

The wearing of a respirator over the mouth and nose is economical and effective but inconvenient.

Desensitization (hyposensitization, immunization) requires elaborate tests to select an effective antigen extract. Nevertheless, it cannot be reliably predicted whether the treatment will result in clinical improvement. This method is moreover not free of the risk of side effects hazardous for health, such as, for example, asthmatoid and urticarial symptoms. Furthermore, the desensitization treatments require a relatively large expenditure of time and money.

Symptomatic treatment of the allergic reaction with oral antihistamines to reduce swelling of the mucous membranes is with many of these products associated with the risk of a sedative effect, which has an adverse effect on the patient's wellbeing and speed of reactions. Furthermore, side effects are not seldom, such as, for example, headaches, dry mouth and concentration disorders.

Furthermore, also the use of sympathomimetics, products based on cromoglicic acid and glucocorticoids is also associated with disadvantages because of numerous side effects and/or high costs.

For a detailed description of this prior art, reference is made to DE-C 41 17 887. This relates to the use of saturated hydrocarbons, which are defined pharmaceutically/cosmetically as petrolatum, as nose ointment for the prophylaxis of inhalation allergic reactions, in particular of the “hayfever” type, the petrolatum used therein having the following properties:

congealing point by the DIN 51 556 method: 49 to 52° C.;

viscosity by the DIN 51 562 method: 6 mm²/s (100° C.)

cone penetration by the DIN 51 580 method: 150 to 170;

average chain length of the hydrocarbons in the petrolatum: 26±1 C atoms;

C number range of the hydrocarbons: C₁₅ to C₆₀.

In view of this prior art, the object on which the present invention was based was to provide a further improved nose ointment for a reliable prophylactic effect on inhalation allergic reactions based on a further improved petrolatum. This nose ointment was intended in turn to entail no health risks for the patient, to have a wide range of a spectrum of activities against various allergens in inhaled air, be pleasant to use and finally to cause only low treatment costs.

This and other objects are achieved by the nose ointment according to claim 1, namely by a nose ointment for the prophylaxis of inhalation allergic reactions comprising at least one mixture of saturated hydrocarbons.

In contrast to the petrolatum described in DE-C 41 17 887, the petrolatum which is now used according to the invention has a larger proportion of long-chain saturated hydrocarbons.

There are no restrictions on the petrolatum which can be used according to the invention as long as it complies with the purity requirements of the quality which can be used pharmaceutically/cosmetically according to the DAB purity provision and has the parameters necessary according to the invention in relation to the usual properties.

Thus, the petrolatum used according to the invention has a viscosity at 100° C. by the DIN 51 562 method of >6 mm²/s, preferably >8 mm²/s and, in particular, a viscosity of 8.5 to 15 mm²/s.

In a further preferred embodiment there is use of a petrolatum which has at least one of the following properties (a) to (e):

(a) congealing point (DIN 51 556) 47 to 56° C.

(b) cone penetration by the DIN 51 580 method of from 155 to 185;

(c) maximum 5% by weight of the saturated hydrocarbons have a chain length of <25±2;

(d) C number range of the saturated hydrocarbons (GC determination): comprises C₁₉ to C₅₂, in each case ±2; and

(e) number average molecular weight: >400, preferably >480 and, in particular >500 g/mol.

Care additives for ointments which can be employed are all additives known for this purpose, preferably silicones, panthenol, lanolin, lecithin, dexpanthenol, provitamin B5, pantothenic acid, panthenyl ethyl ether, panthenyl ethyl ether acetate, panthenyl triacetate, allantoin, vegetable (algae, lichen) oils, such as, for example, almond oil, wheatgerm oil, avocado oil, waxes such as, for example, paraffins, extracts, preferably CO₂ extracts, such as, for example, camomile, sea buckthorn and calendula. In particular heliotropin can be used as care additive.

The nose ointment according to the invention can be applied with the finger or with an applicator. However, the site of application in the nose is essential for the protective effect against inhalation allergic reactions. To achieve reliable prophylaxis it must be ensured that the inner walls of the nose are covered with the nose ointment in the region of the two nasal valves.

If the nose ointment according to the invention is applied only at the lower end of the nasal vestibule in the region of the outer nostrils, it is impossible to preclude the inhalation allergic reaction. However, when used correctly, it is possible with the aid of the nose ointment according to the invention to achieve partial avoidance of exposure to inhaled allergens. Since the nose ointment according to the invention is unable, owing to its particular physical properties, to penetrate into the nasal mucous membranes and is not absorbed, but remains in place as a protective film, a mechanical barrier is produced against the allergen carriers picked up and transported by the respiratory flow. It is possible in this way very substantially to prevent the allergic reaction.

As already described in DE-C 41 17 887, an essential advantage of the present invention is that allergic reactions do not occur, without the need to expose the patient's metabolism to systemic active substances. The main ingredients of the nose ointment, the saturated hydrocarbons mentioned, show inert behaviour and are not absorbed. This means that the nose ointment according to the invention can be employed without risk. In addition, there is no decline in the effect due to the patient's body becoming habituated either. The ability to perceive odours and, coupled therewith, taste remains unimpaired.

Further advantages of the nose ointment according to the invention which may be mentioned are:

comparatively low costs;

patient can carry out the preventive treatment himself;

application of the nose ointment according to the invention with addition has a simultaneous care action.

Furthermore, the nose ointment according to the invention is able not only to prevent the swelling, outflow of secretion and itching in the interior of the nose on exposure to allergen, but also to eliminate the irritation reactions in the eye and throat regions.

Accordingly, the present invention also relates to the use of at least one mixture, defined pharmaceutically/cosmetically as petrolatum, of saturated hydrocarbons as defined in any of claims 1 to 5 in or as nose ointment(s) for the prophylaxis of inhalation allergic reactions, wherein particularly the atopic rhinitis of the “hayfever” type is affected with a beneficial effect.

In conclusion, it may also be mentioned that, because of the great efficiency of the nose ointment according to the invention and of the use according to the invention, a long-lasting, reliable protective effect is achieved, although very small amounts are needed only. With amounts of a few milligrams, which are spread in front of the nasal valves, it is possible to achieve freedom from symptoms for several hours, even with a high pollen count, without reapplication of the nose ointment according to the invention being necessary.

1. Nose ointment for the prophylaxis of inhalation allergic reactions comprising at least one mixture of saturated hydrocarbons
characterized in that
the at least one mixture of saturated hydrocarbons has a viscosity by the DIN 51 562 method of >6 mm²/s (100° C.).

2. Nose ointment according to claim 1, where the at least one mixture of saturated hydrocarbons has less than 20% by weight of linear saturated hydrocarbons (n-paraffins).

3. Nose ointment according to claim 1 or 2, wherein the at least one mixture of saturated hydrocarbons exhibits at least one of the following properties:
(a) congealing point (ISO 2207) 47 to 56° C.
(b) cone penetration by the DIN 51 580 method of from 155 to 185;
(c) maximum 5% by weight of the saturated hydrocarbons have a chain length of <25±2;
(d) C number range of the saturated hydrocarbons (GC determination): comprises C₁₉ to C₅₂, in each case ±2; and
(e) number average molecular weight: >400, preferably >480 and, in particular >500 g/mol.

4. Nose ointment according to any of the claims 1 to 3, which additionally contains at least one care additive.

5. Nose ointment according to claim 4, wherein the care additive is selected from heliotropin, silicones, panthenol, lanolin, lecithin, dexpanthenol, provitamin B5, pantothenic acid, panthenyl ethyl ether, panthenyl ethyl ether acetate, panthenyl triacetate, allantoin, almond oil, wheatgerm oil, avocado oil, paraffins and CO₂ extracts of camomile, sea buckthorn or calendula, and mixtures of two or more thereof.

6. Use of at least one mixture, which is defined pharmaceutically/cosmetically as petrolatum, of saturated hydrocarbons as defined in any of claims 1 to 5 in or as nose ointment(s) for the prophylaxis of inhalation allergic reactions.

7. Use according to claim 6, wherein the inhalation allergic reaction is an atopic rhinitis of the “hayfever” type.
